(19) United States
(12) Patent Application Publication
(10) Pub. No.: US 2007/0179222 A1
(43) Pub. Date: Aug. 2, 2007

(54) Tetrabenzodiazadiketoperylene pigments and dyes

(76) Inventors: Damien Thurber Cole, Drexel Hill, PA (US); Joseph E. Sarver, Erial, NJ (US); Colin Dennis Campbell, Claymont, DE (US)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 11/640,805

(22) Filed: Dec. 18, 2006

Related U.S. Application Data
(63) Continuation-in-part of application No. 11/589,530, filed on Oct. 30, 2006.
(60) Provisional application No. 60/738,455, filed on Nov. 21, 2005.

(51) Int. Cl. C07D 471/02; C08K 5/34

(52) U.S. Cl. 524/90; 546/28

(57) ABSTRACT

Novel tetrabenzodiazadiketoperylene dyes and pigment and methods of coloring polymer compositions are described.

This application is a continuation-in-part U.S. application Ser. No. 11/589,530 filed Oct. 30, 2006.

Novel tetrabenzodiazadiketoperylene compounds, useful as pigments and dyes for polymeric substrates are disclosed. These compounds are also valuable in applications including laser marking, IR reflectance and laser welding.

Copending U.S. application Ser. No. 11/589,530, incorporated herein in its entirety by reference, discloses tetrabenzodiazadiketoperylene pigment compositions which when exposed to laser marking conditions, produce markings that are visible under UV light, but not visible under ambient visible light.

Copending U.S. application Ser. No. 11/589,585, incorporated herein in its entirety by reference, discloses tetrabenzodiazadiketoperylene pigment compositions which reflect large portions of the infrared spectrum useful in preventing heat build up, but the compositions are transparent to other portions of the infra red spectrum making them useful in laser welding applications.

Laser marking is a well known and important means for quickly and cleanly inscribing plastic surfaces with identification marks, such as date codes, batch codes, bar codes or part numbers, functional marks, such as computer keyboard characters, and decorative marks, such as company logos. Common laser marks are a dark mark on a lighter colored background or a light mark on a dark colored background. However, colored laser marks on plastic articles, such as electronic components, exterior automotive parts, utensils, and the like, are also desirable in order to eliminate the cost and environmental complications associated with inks, masks, and other printing or hot-stamping methods now used for color imprinting.

U.S. Pat. No. 4,861,620, incorporated herein in its entirety by reference, discloses pigments that undergo a change of internal structure and hence color due to a temperature increase by laser irradiation.

U.S. Pat. No. 6,022,905, incorporated herein in its entirety by reference, discloses a laser-marked plastic article comprising at least two differently colored laser marks and a method for producing the article by exposing to various laser energies a thermoplastic composition comprising a laser energy absorbing additive and color pigments capable of chemically changing color at higher than a predetermined temperature.

U.S. Pat. No. 6,372,394, incorporated herein in its entirety by reference, relates to a method of marking articles by a laser and more particularly to a method of marking security documents or other documents having a clear substrate covered by opacifying layers.

U.S. Pat. No. 5,028,643, incorporated herein in its entirety by reference, discloses a tetrabenzodiazadiketoperylene pigment and a method for its preparation. Only one compound is disclosed, claimed or exemplified.

Other novel tetrabenzodiazadiketoperylenes, also useful in a variety of applications, are provided by the present invention.

### DESCRIPTION OF THE INVENTION

A tetrabenzodiazadiketoperylene of formula I is provided

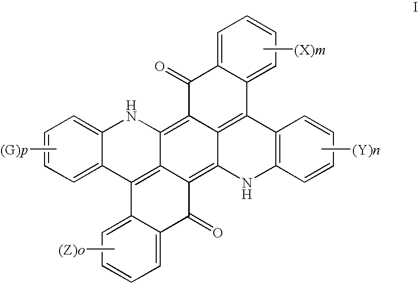

wherein

X, Y, Z and G independently of each other are

C₁₋₁₂ alkyl, C₃₋₆ cycloalkyl, C₇₋₁₂ aralkyl, C₆₋₁₀ aryl, C₃₋₉ saturated or unsaturated heterocycle, halogen, —OR, CF₃, —COOR,—CONR′R, NO₂, NR′R, SO₃H or SO₂NR′R;

R and R′, independently of each other are hydrogen, C₁₋₈ alkyl, C₃₋₆ cycloalkyl, C₆₋₁₀ aromatic or C₇₋₁₂ aralkyl;

m, n, o and p are independently 0, 1, 2, 3 or 4, and when m, n, o or p is 2, 3 or 4, each X, Y, Z or G substituent may each be a different group as defined above

with the proviso that at least one of m, n, o and p are 1, 2, 3 or 4.

For example, X, Y, Z and G independently of each other are C₁₋₁₂ alkyl, C₇₋₁₂ aralkyl, C₆₋₁₀ aryl, F, Cl, Br, I, OR, COOR, CONR′R, NO₂, NR′R, SO₃H or SO₂NR′R.

For example, X, Y, Z and G independently of each other are C₁₋₁₂ alkyl, C₇₋₁₂ aralkyl, C₆₋₁₀ aryl, F, Cl, Br, OR, COOR,CONR′R, NO₂, and m, n, o and p are independently 0, 1 or 2 with the proviso that at least one of m, n, o and p are 1 or 2.

For example, X, Y, Z and G independently of each other are C₁₋₁₂ alkyl, Br, Cl, OR, NO₂, and m, n, o and p are independently selected from 0, 1 or 2 provided that at least one of m, n, o and p are 1 or 2.

For example, the tetrabenzodiazadiketoperylene I is a compound of the formula

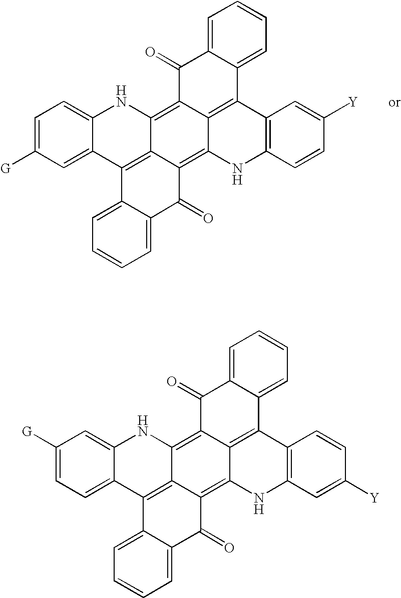

wherein G and Y are defined as above, for example, G and Y are independently selected from C₁₋₈ alkyl, Cl, Br, NO₂, OH and OC₁₋₈ alkyl;

for example

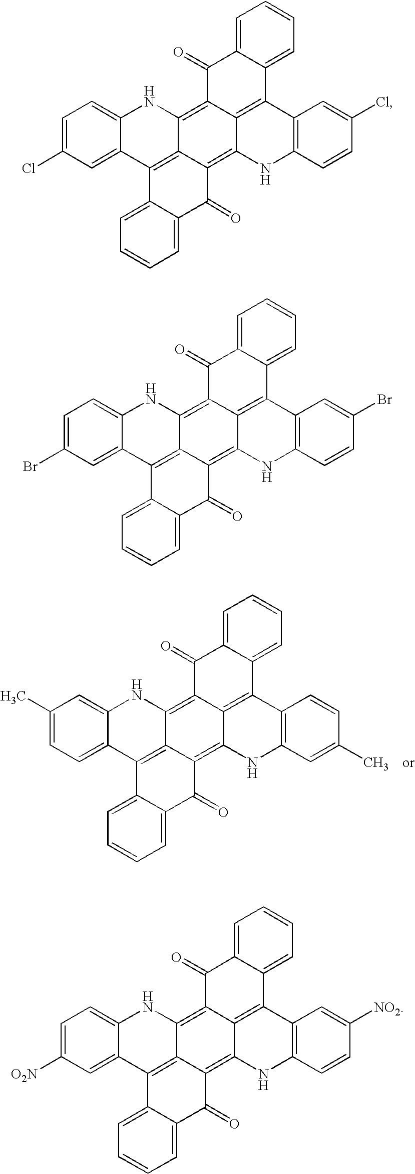

The tetrabenzodiazadiketoperylenes may be symmetrical or unsymmetrical.

Alkyl is straight chain or branched alkyl of the specified number of carbon atoms and is for example methyl, ethyl, n-propyl, n-butyl, sec-butyl, tert-butyl, n-hexyl, n-octyl, 2-ethylhexyl, n-nonyl, n-decyl, n-undecyl, n-dodecyl.

C₃₋₉ saturated or unsaturated heterocycle is a substituted or unsubstituted monocyclic or polycyclic ring of at least 5 atoms, containing 3-9 carbon atoms which heterocycle may also be ionically charged.

For example, saturated or unsaturated heterocycle is a 5, 6, or 7 membered ring containing 1, 2 or 3 nitrogen atoms which may be fused to another carbocyclic or heterocyclic ring.

C₇₋₁₂ aralkyl is for example benzyl, methyl benzyl, cumyl.

The tetrabenzodiazadiketoperylenes of the invention form pigment particles which may be processed using any known method such as milling, grinding, surface treatment etc. Additives commonly encountered in such processing steps may also be present which disclosures are incorporated herein by reference, and may also become part of the finished pigment composition, see for example U.S. Pat. Nos. 5,194,088; 5,997,627 and 7,122,081, which disclosures are incorporated herein by reference.

Such pigments are useful in coloring natural and synthetic polymer substrates and are incorporated therein using standard methods such as extrusion, blending, dispersion, Brabender mixing etc.

In certain polymers, the tetrabenzodiazadiketoperylenes may be soluble and can be incorporated as dyes using any known method common to coloration including dissolution.

The tetrabenzodiazadiketoperylenes is present in the substrate in an “effective amount”, that is an amount that provides the desired coloration effect. For example, the tetrabenzodiazadiketoperylene pigment or dye is present in a polymer composition in an amount of as little as 0.01 to 15% weight percent based on the total weight of the composition, for example 0.1 to 10% based on the total weight of the composition, but can be present in much higher amounts, for example as high as 50% to 99% especially when used in a coating or impregnated into the surface of an article.

Typically the tetrabenzodiazadiketoperylene of the invention is present in a polymer composition in amounts of from about 0.01% to about 15%, for example, from about 0.1% to about 10%, for example, from about 0.1% to about 5%, all percentages being weight percent based on the total weight of the polymer composition.

Other dyes or pigments, including effect pigments may be present and more than one tetrabenzodiazadiketoperylene may be used.

Accordingly, the amount of polymer in a composition may be from 1 to 99.999% by weight, based on the total weight of the composition.

The polymer composition may also comprise further components, such as antioxidants, UV absorbers, hindered amine or other light stabilizers, phosphites or phosphonites, benzofuran-2-ones, thiosynergists, polyamide stabilizers, metal stearates, nucleating agents, fillers, reinforcing agents, lubricants, emulsifiers, dyes, pigments, dispersents, optical brighteners, flame retardants, antistatic agents, blowing agents and the like, in amounts of, for example, from 0.001 to 90% by weight of further components, based on the total weight of the composition.

The naturally occurring or synthetic polymer containing the tetrabenzodiazadiketoperylene dye or pigment may be, for example, a thermoplastic, elastomeric, thermoset, crosslinked or inherently crosslinked polymer. The polymer may be a co-polymer, a polymer blend or part of a composite.

Examples of thermoplastic, thermoset, elastomeric, inherently crosslinked or crosslinked polymers include

1. Polymers of mono- and di-olefins, for example polypropylene, polyisobutylene, polybutene-1, poly-4-methylpentene-1, polyisoprene or polybutadiene and also polymerisates of cyclo-olefins, for example of cyclopentene or norbornene; and also polyethylene (which may optionally be crosslinked), for example high density polyethylene (HDPE), high density polyethylene of high molecular weight (HDPE-HMW), high density polyethylene of ultra-high molecular weight (HDPE-UHMW), medium density polyethylene (MDPE), low density polyethylene (LDPE), and linear low density polyethylene (LLDPE), (VLDPE) and (ULDPE).

Polyolefins, that is to say polymers of mono-olefins, as mentioned by way of example in the preceding paragraph, especially polyethylene and polypropylene, can be prepared by various processes, especially by the following methods:

a) by free radical polymerisation (usually at high pressure and high temperature);

b) by means of a catalyst, the catalyst usually containing one or more metals of group IVb, Vb, VIb or VIII. Those metals generally have one or more ligands, such as oxides, halides, alcoholates, esters, ethers, amines, alkyls, alkenyls and/or aryls, which may be either π- or σ-coordinated. Such metal complexes may be free or fixed to carriers, for example to activated magnesium chloride, titanium(III) chloride, aluminium oxide or silicon oxide. Such catalysts may be soluble or insoluble in the polymerisation medium. The catalysts can be active as such in the polymerisation or further activators may be used, for example metal alkyls, metal hydrides, metal alkyl halides, metal alkyl oxides or metal alkyl oxanes, the metals being elements of group(s) Ia, IIa and/or IIIa. The activators may have been modified, for example, with further ester, ether, amine or silyl ether groups.

2. Mixtures of the polymers mentioned under 1), for example mixtures of poly-propylene with polyisobutylene, polypropylene with polyethylene (for example PP/HDPE, PP/LDPE) and mixtures of different types of polyethylene (for example LDPE/HDPE).

3. Copolymers of mono- and di-olefins with one another or with other vinyl monomers, for example ethylene/propylene copolymers, linear low density polyethylene (LLDPE) and mixtures thereof with low density polyethylene (LDPE), propylene/butene-1 copolymers, propylene/isobutylene copolymers, ethylene/butene-1 copolymers, ethylene/hexene copolymers, ethylene/methylpentene copolymers, ethylene/heptene copolymers, ethylene/octene copolymers, propylene/butadiene copolymers, isobutylene/isoprene copolymers, ethylene/alkyl acrylate copolymers, ethylene/alkyl methacrylate copolymers, ethylene/vinyl acetate copolymers and copolymers thereof with carbon monoxide, or ethylene/acrylic acid copolymers and salts thereof (ionomers), and also terpolymers of ethylene with propylene and a diene, such as hexadiene, dicyclopentadiene or ethylidenenorbornene; and also mixtures of such copolymers with one another or with polymers mentioned under 1), for example polypropylene-ethylene/propylene copolymers, LDPE-ethylene/vinyl acetate copolymers, LDPE-ethylene/acrylic acid copolymers, LLDPE-ethylene/vinyl acetate copolymers, LLDPE-ethylene/acrylic acid copolymers and alternately or randomly structured polyalkylene-carbon monoxide copolymers and mixtures thereof with other polymers, for example polyamides.

4. Hydrocarbon resins (for example C₅-C₉) including hydrogenated modifications thereof (for example tackifier resins) and mixtures of polyalkylenes and starch.

5. Polystyrene, poly(p-methylstyrene), poly(α-methylstyrene).

6. Copolymers of styrene or α-methylstyrene with dienes or acrylic derivatives, for example styrene/butadiene, styrene/acrylonitrile, styrene/alkyl methacrylate, styrene/-butadiene/alkyl acrylate and methacrylate, styrene/maleic anhydride, styrene/acrylo-nitrile/methyl acrylate; high-impact-strength mixtures consisting of styrene copolymers and another polymer, for example a polyacrylate, a diene polymer or an ethylene/-propylene/diene terpolymer; and also block copolymers of styrene, for example styrene/-butadiene/styrene, styrene/isoprene/styrene, styrene/ethylene-butylene/styrene or styrene/ethylene-propylene/styrene.

7. Graft copolymers of styrene or α-methylstyrene, for example styrene on poly-butadiene, styrene on polybutadiene/styrene or polybutadiene/acrylonitrile copolymers, styrene and acrylonitrile (or methacrylonitrile) on polybutadiene; styrene, acrylonitrile and methyl methacrylate on polybutadiene; styrene and maleic anhydride on polybutadiene; styrene, acrylonitrile and maleic anhydride or maleic acid imide on polybutadiene; styrene and maleic acid imide on polybutadiene, styrene and alkyl acrylates or alkyl methacrylates on polybutadiene, styrene and acrylonitrile on ethylene/propylene/diene terpolymers, styrene and acrylonitrile on polyalkyl acrylates or polyalkyl methacrylates, styrene and acrylonitrile on acrylate/butadiene copolymers, and mixtures thereof with the copolymers mentioned under 6), such as those known, for example, as so-called ABS, MBS, ASA or AES polymers.

8. Halogen-containing polymers, for example polychloroprene, chlorinated rubber, chlorinated and brominated copolymer of isobutylene/isoprene (halobutyl rubber), chlorinated or chlorosulfonated polyethylene, copolymers of ethylene and chlorinated ethylene, epichlorohydrin homo- and co-polymers, especially polymers of halogen-containing vinyl compounds, for example polyvinyl chloride, polyvinylidene chloride, polyvinyl fluoride, polyvinylidene fluoride; and copolymers thereof, such as vinyl chloride/vinylidene chloride, vinyl chloride/vinyl acetate or vinylidene chloride/vinyl acetate.

9. Polymers derived from α,β-unsaturated acids and derivatives thereof, such as polyacrylates and polymethacrylates, or polymethyl methacrylates, polyacrylamides and polyacrylonitriles impact-resistant-modified with butyl acrylate.

10. Copolymers of the monomers mentioned under 9) with one another or with other unsaturated monomers, for example acrylonitrile/butadiene copolymers, acrylo-nitrile/alkyl acrylate copolymers, acrylonitrile/alkoxyalkyl acrylate copolymers, acrylonitrile/vinyl halide copolymers or acrylonitrile/alkyl methacrylate/butadiene terpolymers.

11. Polymers derived from unsaturated alcohols and amines or their acyl derivatives or acetals, such as polyvinyl alcohol, polyvinyl acetate, stearate, benzoate or maleate, polyvinylbutyral, polyallyl phthalate, polyallylmelamine; and the copolymers thereof with olefins mentioned in Point 1.

12. Homo- and co-polymers of cyclic ethers, such as polyalkylene glycols, poly-ethylene oxide, polypropylene oxide or copolymers thereof with bisglycidyl ethers.

13. Polyacetals, such as polyoxymethylene, and also those polyoxymethylenes which contain comonomers, for example ethylene oxide; polyacetals modified with thermoplastic polyurethanes, acrylates or MBS.

14. Polyphenylene oxides and sulfides and mixtures thereof with styrene polymers or polyamides.

15. Polyurethanes derived from polyethers, polyesters and polybutadienes having terminal hydroxyl groups on the one hand and aliphatic or aromatic polyisocyanates on the other hand, and their initial products.

16. Polyamides and copolyamides derived from diamines and dicarboxylic acids and/or from aminocarboxylic acids or the corresponding lactams, such as polyamide 4, polyamide 6, polyamide 6/6, 6/10, 6/9, 6/12, 4/6, 12/12, polyamide 11, polyamide 12, aromatic polyamides derived from m-xylene, diamine and adipic acid; polyamides prepared from hexamethylenediamine and iso- and/or tere-phthalic acid and optionally an elastomer as modifier, for example poly-2,4,4-trimethylhexamethylene terephthalamide or poly-m-phenylene isophthalamide. Block copolymers of the above-mentioned polyamides with polyolefins, olefin copolymers, ionomers or chemically bonded or grafted elastomers; or with polyethers, for example with polyethylene glycol, polypropylene glycol or polytetramethylene glycol. Also polyamides or copolyamides modified with EPDM or ABS; and polyamides condensed during processing (“RIM polyamide systems”).

17. Polyureas, polyimides, polyamide imides, polyether imides, polyester imides, polyhydantoins and polybenzimidazoles.

18. Polyesters derived from dicarboxylic acids and dialcohols and/or from hydroxy-carboxylic acids or the corresponding lactones, such as polyethylene terephthalate, polybutylene terephthalate, poly-1,4-dimethylolcyclohexane terephthalate, polyhydroxy-benzoates, and also block polyether esters derived from polyethers with hydroxyl terminal groups; and also polyesters modified with polycarbonates or MBS.

19. Polycarbonates and polyester carbonates.

20. Polysulfones, polyether sulfones and polyether ketones.

21. Crosslinked polymers derived from aldehydes on the one hand and phenols, urea or melamine on the other hand, such as phenol-formaldehyde, urea-formaldehyde and melamine-formaldehyde resins.

22. Drying and non-drying alkyd resins.

23. Unsaturated polyester resins derived from copolyesters of saturated and unsaturated dicarboxylic acids with polyhydric alcohols, and also vinyl compounds as crosslinking agents, and also the halogen-containing, difficultly combustible modifications thereof.

24. Crosslinkable acrylic resins derived from substituted acrylic esters, e.g. from epoxy acrylates, urethane acrylates or polyester acrylates.

25. Alkyd resins, polyester resins and acrylate resins that are crosslinked with melamine resins, urea resins, isocyanates, isocyanurates, polyisocyanates or epoxy resins.

26. Crosslinked epoxy resins derived from aliphatic, cycloaliphatic, heterocyclic or aromatic glycidyl compounds, e.g. products of bisphenol-A diglycidyl ethers, bisphenol-F diglycidyl ethers, that are crosslinked using customary hardeners, e.g. anhydrides or amines with or without accelerators.

27. Natural polymers, such as cellulose, natural rubber, gelatin, or polymer-homologously chemically modified derivatives thereof, such as cellulose acetates, propionates and butyrates, and the cellulose ethers, such as methyl cellulose; and also colophonium resins and derivatives.

28. Mixtures (polyblends) of the afore-mentioned polymers, for example PP/EPDM, polyamide/EPDM or ABS, PVC/EVA, PVC/ABS, PVC/MBS, PC/ABS, PBTP/ABS, PC/ASA, PC/PBT, PVC/CPE, PVC/acrylates, POM/thermoplastic PUR, PC/thermo-plastic PUR, POM/acrylate, POM/MBS, PPO/HIPS, PPO/PA 6.6 and copolymers, PA/HDPE, PA/PP, PA/PPO, PBT/PC/ABS or PBT/PET/PC.

The thermoplastic, elastomeric, crosslinked or inherently crosslinked polymer is, for example, a polyolefin, polyamide, polyurethane, polyacrylate, polyacrylamide, polyvinyl alcohol, polycarbonate, polystyrene, polyester, polyacetal, a natural or synthetic rubber or a halogenated vinyl polymer such as PVC.

The polymer may be, for example, in the form of a film, sheet, injection-moulded article, extruded workpiece, fiber, laminate, felt or woven fabric. The polymer may also be part of a coating composition.

The tetrabenzodiazadiketoperylene pigments and dyes of the invention may be incorporated into polymer resins according a variety of known methods. For example, they may be added as an individual component during blending, for example, dry blending of the resin prior to processing, or they may be added as a blend, master batch, flush, or other concentrate in another substance prior to processing. The tetrabenzodiazadiketoperylene pigments and dyes may also be added during processing steps. Standard process steps for polymer resins are well known in the literature and include extrusion, coextrusion, compression molding, Brabender melt processing, film formation, injection molding, blow molding, other molding and sheet forming processes, fiber formation etc.

When the tetrabenzodiazadiketoperylene pigments and dyes are used in a film, the film may be a stand alone film or a film that is applied to a surface by, for example, the use of an adhesive, or co-extruded onto the surface. A preformed film may also be applied with heat which includes calendaring, melt applications and shrink wrapping.

The tetrabenzodiazadiketoperylene pigments and dyes are incorporated, for example, via dry blending, surface impregnation, suspension, dispersion, dissolution and other methods known in coatings technology.

Typically, a coating comprises a polymeric binder which can in principle be any binder customary in industry, for example those described in Ullmann's Encyclopedia of Industrial Chemistry, 5th Edition, Vol. A18, pp. 368-426, VCH, Weinheim 1991. In general, it is a film-forming binder based on a thermoplastic or thermosetting resin, predominantly on a thermosetting resin. Examples thereof are alkyd, acrylic, acrylamide, polyester, styrenic, phenolic, melamine, epoxy and polyurethane resins.

For example, non-limiting examples of common coating binders useful in the present invention include silicon containing polymers, fluorinated polymers, unsaturated polyesters, unsaturated polyamides, polyimides, crosslinkable acrylic resins derived from substituted acrylic esters, e.g. from epoxy acrylates, urethane acrylates, polyester acrylates, polymers of vinyl acetate, vinyl alcohol and vinyl amine. The coating binder polymers may be co-polymers, polymer blends or composites.

Coatings are frequently crosslinked with, for example, melamine resins, urea resins, isocyanates, isocyanurates, polyisocyanates, epoxy resins, anhydrides, poly acids and amines, with or without accelerators.

The binder can be a cold-curable or hot-curable binder; the addition of a curing catalyst may be advantageous. Suitable catalysts which accelerate curing of the binder are described, for example, in Ullmann's Encyclopedia of Industrial Chemistry, Vol. A18, p.469, VCH Verlagsgesellschaft, Weinheim 1991.

The binder may be a surface coating resin which dries in the air or hardens at room temperature. Exemplary of such binders are nitrocellulose, polyvinyl acetate, polyvinyl chloride, unsaturated polyester resins, polyacrylates, polyurethanes, epoxy resins, phenolic resins, and especially alkyd resins. The binder may also be a mixture of different surface coating resins. Provided the binders are curable binders, they are normally used together with a hardener and/or accelerator.

Examples of coating compositions containing specific binders are:

1. coatings based on cold- or hot-crosslinkable alkyd, acrylate, polyester, epoxy or melamine resins or mixtures of such resins, if desired with addition of a curing catalyst;

2. two-component polyurethane coatings based on hydroxyl-containing acrylate, polyester or polyether resins and aliphatic or aromatic isocyanates, isocyanurates or polyisocyanates;

3. one-component polyurethane coatings based on blocked isocyanates, isocyanurates or polyisocyanates which are deblocked during baking, if desired with addition of a melamine resin;

4. one-component polyurethane coatings based on a Trisalkoxycarbonyltriazine crosslinker and a hydroxyl group containing resin such as acrylate, polyester or polyether resins;

5. one-component polyurethane coatings based on aliphatic or aromatic urethaneacrylates or polyurethaneacrylates having free amino groups within the urethane strukture and melamine resins or polyether resins, if necessary with curing catalyst;

6. two-component coatings based on (poly)ketimines and aliphatic or aromatic isocyanates, isocyanurates or polyisocyanates;

7. two-component coatings based on (poly)ketimines and an unsaturated acrylate resin or a polyacetoacetate resin or a methacrylamidoglycolate methyl ester;

8. two-component coatings based on carboxyl- or amino-containing polyacrylates and polyepoxides;

9. two-component coatings based on acrylate resins containing anhydride groups and on a polyhydroxy or polyamino component;

10. two-component coatings based on acrylate-containing anhydrides and polyepoxides;

11. two-component coatings based on (poly)oxazolines and acrylate resins containing anhydride groups, or unsaturated acrylate resins, or aliphatic or aromatic isocyanates, isocyanurates or polyisocyanates;

12. two-component coatings based on unsaturated polyacrylates and polymalonates;

13. thermoplastic polyacrylate coatings based on thermoplastic acrylate resins or externally crosslinking acrylate resins in combination with etherified melamine resins;

14. paint systems based on siloxane-modified or fluorine-modified acrylate resins.

The coating composition can also comprise further components, examples being solvents, pigments, dyes, plasticizers, stabilizers, thixotropic agents, drying catalysts and/or levelling agents. Examples of possible components are those described in Ullmann's Encyclopedia of Industrial Chemistry, 5th Edition, Vol. A18, pp. 429-471, VCH, Weinheim 1991.

Possible drying catalysts or curing catalysts are, for example, organometallic compounds, amines, amino-containing resins and/or phosphines. Examples of organometallic compounds are metal carboxylates, especially those of the metals Pb, Mn, Co, Zn, Zr or Cu, or metal chelates, especially those of the metals Al, Ti or Zr, or organometallic compounds such as organotin compounds, for example.

Examples of metal carboxylates are the stearates of Pb, Mn or Zn, the octoates of Co, Zn or Cu, the naphthenates of Mn and Co or the corresponding linoleates, resinates or tallates.

Examples of metal chelates are the aluminium, titanium or zirconium chelates of acetylacetone, ethyl acetylacetate, salicylaldehyde, salicylaldoxime, o-hydroxyacetophenone or ethyl trifluoroacetylacetate, and the alkoxides of these metals.

Examples of organotin compounds are dibutyltin oxide, dibutyltin dilaurate or dibutyltin dioctoate.

Examples of amines are, in particular, tertiary amines, for example tributylamine, triethanolamine, N-methyldiethanolamine, N-dimethylethanolamine, N-ethylmorpholine, N-methylmorpholine or diazabicyclooctane (triethylenediamine) and salts thereof. Further examples are quaternary ammonium salts, for example trimethylbenzyl-ammonium chloride.

Amino-containing resins are simultaneously binder and curing catalyst. Examples thereof are amino-containing acrylate copolymers.

The curing catalyst used can also be a phosphine, for example triphenylphosphine.

The coating compositions can also be radiation-curable coating compositions. In this case, the binder essentially comprises monomeric or oligomeric compounds containing ethylenically unsaturated bonds, which after application are cured by actinic radiation, i.e. converted into a crosslinked, high molecular weight form. Where the system is UV-curing, it generally contains a photoinitiator as well. Corresponding systems are described in the abovementioned publication Ullmann's Encyclopedia of Industrial Chemistry, 5th Edition, Vol. A18, pages 451-453. In radiation-curable coating compositions, the novel stabilizers can also be employed without the addition of sterically hindered amines.

The coating may also be a radiation-curable, solvent-free formulation of photopolymerisable compounds. Illustrative examples are mixtures of acrylates or methacrylates, unsaturated polyester/styrene mixtures or mixtures of other ethylenically unsaturated monomers or oligomers.

The coating compositions can comprise an organic solvent or solvent mixture in which the binder is soluble. The coating composition can otherwise be an aqueous solution or dispersion. The vehicle can also be a mixture of organic solvent and water. The coating composition may be a high-solids paint or can be solvent-free (e.g. a powder coating material). Powder coatings are, for example, those described in Ullmann's Encyclopedia of Industrial Chemistry, 5th Ed., A18, pages 438-444. The powder coating material may also have the form of a powder-slurry (dispersion of the powder preferably in water).

When used in a coating, the compounds of formula I are incorporated into the coating via techniques common in the art.

The coating composition can be applied to any desired substrate, for example to metal, wood, plastic, composite, glass or ceramic material substrates by the customary methods, for example by brushing, spraying, pouring, draw down, spin coating, dipping or electrophoresis; see also Ullmann's Encyclopedia of Industrial Chemistry, 5th Edition, Vol. A18, pp.491-500.

Multilayer systems are possible, where the tetrabenzodiazadiketoperylene pigment or dye may reside in a coating or substrate layer which is then itself coated with another coating, such as a protective coating.

The tetrabenzodiazadiketoperylene compounds of formula I are prepared by known methods. For example, the tetrabenzodiazadiketoperylene compounds of formula I are prepared by reacting a mixture of an appropriately substituted 2-aminobenzophenone with a dialkyldisuccinylsuccinate to form an intermediate which is then heated in a high boiling solvent. The substituted 2-aminobenzophenones and dialkyldisuccinylsuccinate are items of commerce or are readily prepared from items of commerce using known methods. Commercially available substituted 2-aminobenzophenones include 2-amino-5-bromobenzophenone, 2-amino-5-chlorobenzophenone, 2-amino-5-nitrobenzophenone and 2-amino-4-methylbenzophenone. More details regarding the preparation of the tetrabenzodiazadiketoperylene can be gleaned from the Examples below and from the general procedures in U.S. Pat. No. 5,028,643.

The present invention therefore relates to novel tetrabenzodiazadiketoperylene compounds of formula I as described above;

pigment compositions comprising the novel tetrabenzodiazadiketoperylene compounds; polymer compositions, including plastic articles, films and coatings, comprising the novel tetrabenzodiazadiketoperylene dyes or pigments;

and methods for coloring polymer compositions by incorporating therein as dyes or pigments novel tetrabenzodiazadiketoperylenes of formula I.

### EXAMPLES

The following Examples serve to illustrate the invention without intending to be restrictive in nature; parts and percentages are by weight, unless otherwise stated.

### Example 1

2,12-dibromo-5,15-diaza-5b,15b-dihydrotetrabenzo[a,gj,p]perylene-6,16-dione

To a mixture of 45.76 grams of dimethyldisuccinylsuccinate and 121.71 grams of 2-amino-5-bromobenzophenone in 500 mL of methanol in a 2 L autoclave under nitrogen is added 2 mL concentrated HCl. The resulting mixture is heated at 99-107° C. and 50-57 psi for 7 hours and then allowed cool to room temperature. The reaction product is isolated and washed with ˜1.5 L methanol and dried at 75° C. under 12 in. Hg to yield 89.22 grams of intermediate (a): mp 281-283° C., 285-288° C. (dec.): 1H NMR (500 MHz, CDCl₃) δ 3.62 (s, 6H), 5.37 (s, 2H), 7.21 (m, 2H), 7.43 (m, 2H), 7.56 (m, 8H), 7.78 (dd, 2H), 8.02 (d, 2H).

Intermediate (a), 20.79 grams, is added in small portions over 40 minutes to 600 mL of THERMINOL VP-1 (a high boiling aromatic-ether based solvent) at reflux (257° C.) with vigorous agitation and the resulting solution is refluxed for 1 hour before cooling to ˜105° C. to precipitate the product which is filtered hot and washed with ˜1 L of methanol. The presscake is dried at 75° C. and 12.5 in. Hg to yield 18.17 grams of the title compound which is re-crystallized from concentrated H₂SO₄: mp>300° C. Anal. Calc. for C₃₄H₁₆Br₂N₂O₂: C, 63.38; H, 2.50; N, 4.35. Found: C, 62.12; H, 2.54; N, 4.19.

### Example 2

2,12-dichloro-5,15-diaza-5b,15b-dihydrotetrabenzo[a,gj,p]perylene-6,16-diones

To a mixture of45.6 grams dimethyidisuccinylsuccinate and 101.86 grams 2-amino-5-chlorobenzophenone in 500 mL of methanol in round bottomed flask under nitrogen is added 2 mL concentrated HCl. The resulting mixture is heated at reflux for 5 hours and then allowed cool to room temperature. The reaction product is isolated and washed with ˜1.5 L methanol and dried at 80° C. under 12 in. Hg to yield 102.9 grams of intermediate (b): mp 286° C. (dec.): 1H NMR (500 MHz, CDCl₃) δ 3.62 (s, 6H), 5.38 (s, 2H), 7.21 (m, 2H), 7.43 (m, 2H), 7.56 (m, 8H), 7.65 (dd, 2H), 8.10 (d, 2H).

Intermediate (b), 50.0 grams, is added in small portions over 20 minutes to 600 mL of THERMINOL VP-1 (a high boiling aromatic-ether based solvent) at reflux (257° C.) with vigorous agitation and the resulting solution is refluxed for 0.25 hour before cooling to ˜180° C. to precipitate the product which is filtered hot and washed with ˜400 mL of methanol. The presscake is dried at 50° C. and 12.5 in. Hg to yield 37.5 grams of the title compound. Anal. Calc. for C₃₄H₁₆Cl₂N₂O₂: C, 73.53; H, 2.90; N, 5.04; Cl, 12.77. Found: C, 72.88; H, 2.60; N, 4.99; Cl, 12.94.

What is claimed:

1. A tetrabenzodiazadiketoperylene of formula I 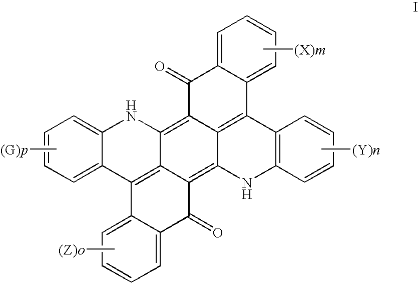
wherein
X, Y, Z and G independently of each other are
C₁₋₁₂ alkyl, C₃₋₆ cycloalkyl, C₇₋₁₂ aralkyl, C₆₋₁₀ aryl, C₃₋₉ saturated or unsaturated heterocycle, halogen, —OR, CF₃, —COOR,—CONR′R, NO₂, NR′R, SO₃H or SO₂NR′R;
R and R′, independently of each other are hydrogen, C₁₋₈ alkyl, C₃₋₆ cycloalkyl, C₆₋₁₀ aromatic or C₇₋₁₂ aralkyl;
m, n, o and p are independently 0, 1, 2, 3 or 4, and when m, n, o or p is 2, 3 or 4, each X, Y, Z or G substituent may each be a different group as defined above
with the proviso that at least one of m, n, o and p are 1, 2, 3 or 4.

2. A tetrabenzodiazadiketoperylene according to claim 1, wherein X, Y, Z and G independently of each other are C₁₋₁₂ alkyl, C₇₋₁₂ aralkyl, C₆₋₁₀ aryl, F, Cl, Br, I, OR, COOR, CONR′R, NO₂, NR′R, SO₃H or SO₂NR′R.

3. A tetrabenzodiazadiketoperylene according to claim 2, wherein X, Y, Z and G independently of each other are C₁₋₁₂ alkyl, C₇₋₁₂ aralkyl, C₆₋₁₀ aryl, F, Cl, Br, OR, COOR,CONR′R, NO₂, and m, n, o and p are independently 0, 1 or 2 with the proviso that at least one of m, n, o and p are 1 or 2.

4. A tetrabenzodiazadiketoperylene according to claim 3, wherein X, Y, Z and G independently of each other are C₁₋₁₂ alkyl, Br, Cl, OR, NO₂, and m, n, o and p are independently selected from 0, 1 or 2 with the proviso that at least one of m, n, o and p are 1 or 2.

5. A tetrabenzodiazadiketoperylene according to claim 1 which is of the formula

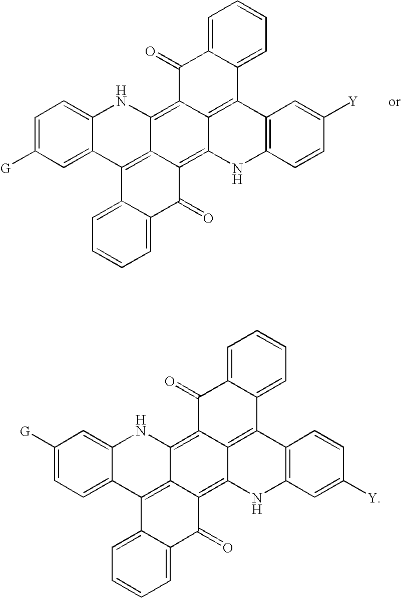

6. A tetrabenzodiazadiketoperylene according to claim 5, wherein G and Y are independently selected from C₁₋₆ alkyl, Cl, Br, NO₂, OH and OC₁₋₈ alkyl.

7. A pigment composition comprising a tetrabenzodiazadiketoperylene of formula I according to claim 1.

8. A method of coloring a polymer composition which method comprises incorporating into the polymer a dye or pigment comprising a tetrabenzodiazadiketoperylene of formula I 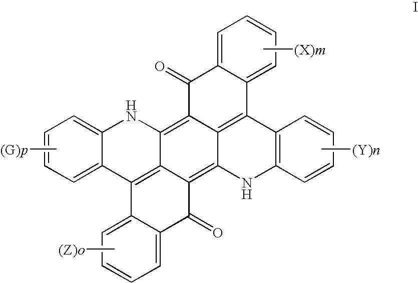
wherein
X, Y, Z and G independently of each other are C₁₋₁₂ alkyl, C₃₋₆ cycloalkyl, C₇₋₁₂ aralkyl, C₆₋₁₀ aryl, C₃₋₉ saturated or unsaturated heterocycle, halogen, —OR, CF₃, —COOR,—CONR′R, NO₂, NR′R, SO₃H or SO₂NR′R;
R and R′, independently of each other are hydrogen, C₁₋₈ alkyl, C₃₋₆ cycloalkyl, C₆₋₁₀ aromatic or C₇₋₁₂ aralkyl;
m, n, o and p are independently 0, 1, 2, 3 or 4, and when m, n, o or p is 2, 3 or 4, each X, Y, Z or G substituent may each be a different group as defined above
with the proviso that at least one of m, n, o and p are 1, 2, 3 or 4.

9. A method according to claim 8, wherein the polymer composition comprises a thermoplastic, elastomeric, crosslinked or inherently crosslinked polymer.

10. A method according to claim 9, wherein the polymer composition is a molded article, film or coating.

11. A method according to claim 8, wherein the dye or pigment comprising a tetrabenzodiazadiketoperylene of formula I is incorporated in to the polymer by blending, extrusion, coextrusion, compression molding, Brabender melt processing, film formation, injection molding, blow molding, fiber formation, surface impregnation, suspension, dispersion or dissolution.
